(19) United States
(12) Patent Application Publication
(10) Pub. No.: US 2011/0057124 A1
(43) Pub. Date: Mar. 10, 2011

(54) RADIATION THERAPY APPARATUS AND METHOD FOR MONITORING AN IRRADIATION

(76) Inventor: Eike Rietzel, Weiterstadt (DE)

(21) Appl. No.: 12/874,944

(22) Filed: Sep. 2, 2010

(30) Foreign Application Priority Data
Sep. 7, 2009 (DE) DE102009040389.2

(51) Int. Cl. A61N 5/00; G01N 23/04

(52) U.S. Cl. 250/492.1; 378/62

(57) ABSTRACT

A radiation therapy apparatus includes a source for providing a therapy beam, an imaging apparatus including an x-ray source and an x-ray detector, and a control apparatus for the imaging apparatus. The control apparatus is configured to read out the x-ray detector during an irradiation of the target object with the therapy beam, in order to record photons, which develop within the target object as a result of the interaction of the therapy beam with the target object.

This application claims the benefit of DE 10 2009 040 389.2 filed Sep. 7, 2009, which is hereby incorporated by reference.

### BACKGROUND

The present embodiments relate to a radiation therapy apparatus.

Particle therapy, which is a special form of radiation therapy, has gained increasing importance over the last few years. Particle therapy enables methods for treating tissue (e.g., tumor diseases) to be implemented. Irradiation methods, like those used in particle therapy, are also used in non-therapeutic fields. Research work for product development is within the scope of particle therapy, the research work being implemented, for example, on non-living phantoms or bodies or being the irradiation of materials.

Charged particles such as protons, carbon ions or other charged particles, for example, are accelerated to high energies, shaped to form a particle beam and fed to one or several irradiation rooms by way of a high energy beam transportation system. The object to be irradiated with a target volume is irradiated in one of these irradiation rooms with the particle beam.

The interaction of the particle beam with the material of the target object to be irradiated produces unstable cores, both in the particles of the particle beam and also in the material of the target object. Positron emission tomography (PET) systems, with which the break-up of these unstable cores may be detected, are known.

### SUMMARY AND DESCRIPTION

The present embodiments may obviate one or more of the drawbacks or limitations in the related art. For example, in one embodiment, a radiation therapy apparatus that enables a simple and cost-effective monitoring of the break-ups of unstable cores may be provided.

In one embodiment, a radiation therapy apparatus includes a source for providing a therapy beam, which may be directed from the source onto a target object to be irradiated, and an imaging apparatus including an x-ray source and an x-ray detector. The x-ray source and the x-ray detector may be arranged such that diagnostic x-rays may be directed from the x-ray source, through the target object to be irradiated and onto the x-ray detector. The radiation therapy apparatus also includes a control apparatus (e.g., a controller) for the imaging apparatus. The control apparatus is configured to read out the x-ray detector during an irradiation of the target object with the therapy beam. As a result, a recording of photons that are produced within the target object as a result of the interaction of the therapy beam with the target object may be implemented.

In the case of radiation therapy apparatuses, a patient or another target object to be irradiated is accurately positioned spatially with respect to the therapy beam in order to provide the desired irradiation. An imaging apparatus that enables the internal structure of the target volume to be irradiated to be mapped in the target object may be used. The position of the target volume may be determined immediately prior to the start of an irradiation and if necessary, may be corrected accordingly. The imaging apparatus may also be used during the irradiation, in order to detect and monitor the position of the target volume (e.g., during a fluoroscopy-type operation).

An already existing imaging apparatus may also be used for another purpose, to record the radioactive break-ups that are produced in the target volume as a result of the interaction of the therapy beam with the material of the target volume by way of the photons emitted therefrom. As the photons generated in this way in the target volume act as a measure for the dose deposited in the target volume, a monitoring of the dose deposition implemented with the therapy beam may be implemented in this way. Additional devices such as, for example, an additional PET system or a SPECT system are not needed. Accordingly, costs and space requirements are reduced. In particular, the photons recorded in this way are evaluated and may be used to control the radiation therapy apparatus (e.g., even during an irradiation session or for subsequent irradiation sessions).

In one embodiment, the radiation therapy apparatus also includes an evaluation apparatus (e.g., a computing unit) that is configured to reconstruct a dose distribution deposited in the target object by the therapy beam at least partially from the photons recorded with the x-ray detector. The deposited dose distribution may, in this way, already be determined at least partially during an irradiation or after an irradiation, so that the irradiation success may be monitored. If the x-ray radiation detector is moved about the target object, for example, a dose distribution may be determined in three dimensions from the recorded photons. However, conclusions may be drawn as to the range of the therapy beam and/or a lateral extension of the deposited dose even with a stationary detector, which only records data from one specific direction,

In one embodiment, the control apparatus is configured to read out the x-ray detector without simultaneously activating the x-ray source. The imaging apparatus may thus be operated in two different modes. In a first mode of operation, the imaging apparatus is operated such that the x-ray detector interacts with the x-ray source and is read out while the x-ray source is activated. By contrast, in a second mode of operation, the x-ray detector may only be used to detect the photons generated in the target object.

In one embodiment, the imaging apparatus may be arranged at different positions relative to the target object. This enables the photons generated in the target object to be recorded from different angles of view or enables a suitable position of the x-ray detector relative to the target object and the beam exit to be selected. Accordingly, the information content of the recording may be improved. This may be useful, for example, with a reconstruction of the deposited dose distribution. The imaging apparatus may be arranged, for example, on a positioning apparatus (e.g., on a robot arm), with which the imaging apparatus may be moved to different positions. The imaging apparatus may be controlled, for example, such that the position of the imaging apparatus for recording the photons generated by the therapy beam may be changed during an irradiation of the target volume, in order, for example, to obtain three-dimensional information about the deposited dose distribution.

In one embodiment, the radiation therapy apparatus includes at least one further imaging apparatus, which includes a further x-ray source and a further x-ray detector. The further imaging apparatus may also be configured and used like the first imaging apparatus. Alternatively, the first imaging apparatus may be operated in the first mode of operation, and the second imaging apparatus may be operated in the second mode of operation, so that a monitoring of the dose deposition and the position of the target volume in the target object may be implemented at the same time.

In one embodiment, the x-ray detector includes an adjustment apparatus for modifying the photons striking the x-ray detector. The adjustment apparatus may be positioned reversibly in front of the x-ray detector and in an automatically controlled fashion. The x-ray detector may be operated for instance in a first, conventional imaging mode without the adjustment apparatus, while the adjustment apparatus in the second operating mode filters and/or modifies the photons generated in the target object by interaction with the therapy beam before the photons strike the x-ray detector.

A method for monitoring an irradiation of a target object includes irradiating the target object with a therapy beam and generating photons in the target object as a result of the interaction of the therapy beam with the target object. The method includes recording the photons using an imaging apparatus that includes an x-ray source and an x-ray detector for x-raying the target object with an x-ray beam. The method also includes evaluating the recorded photons.

The methods of the present embodiments are suitable both within the scope of an irradiation of a patient and also within the scope of an irradiation of a non-human or non-animal body (e.g., when irradiating a phantom for test, research and/or calibration purposes).

In one embodiment, the photons are recorded with the x-ray detector at a point in time at which the x-ray source is not activated.

The recorded photons are evaluated such that a dose distribution deposited in the target object by the therapy beam is at least partially reconstructed from the recorded photons.

The x-ray detector may be moved to a different position during the recording of the photons.

To record the photons, an adjustment apparatus for modifying the photons striking the x-ray detector may be arranged upstream of the x-ray detector. The adjustment apparatus may be positioned reversibly upstream of the x-ray detector and in an automatically controlled fashion.

The x-ray detector may be operated in an imaging mode, in which the x-ray detector interacts with the x-ray source in order to record image data of an object to be irradiated. The x-ray detector may also be operated in a therapy monitoring mode, in which the x-ray detector records the photons produced in the body by interaction with the therapy beam without interaction with the x-ray source.

The preceding and subsequent description of the individual features relates both to the apparatus embodiments and the method embodiments, without this being explicitly explained in detail in each case; the individual features disclosed here may also be of significance to the present embodiments in other combinations than those shown.

### BRIEF DESCRIPTION OF THE DRAWINGS

FIG. 1 shows a particle therapy system;

FIG. 2 shows one embodiment of a radiation therapy apparatus that is operated in the imaging mode;

FIG. 3 shows one embodiment of the radiation therapy apparatus of FIG. 2 in the therapy monitoring mode;

FIG. 4 shows one embodiment of a radiation therapy apparatus including two imaging apparatuses; and

FIG. 5 shows a flow chart of monitoring an irradiation of a target object according to one embodiment.

### DETAILED DESCRIPTION OF THE DRAWINGS

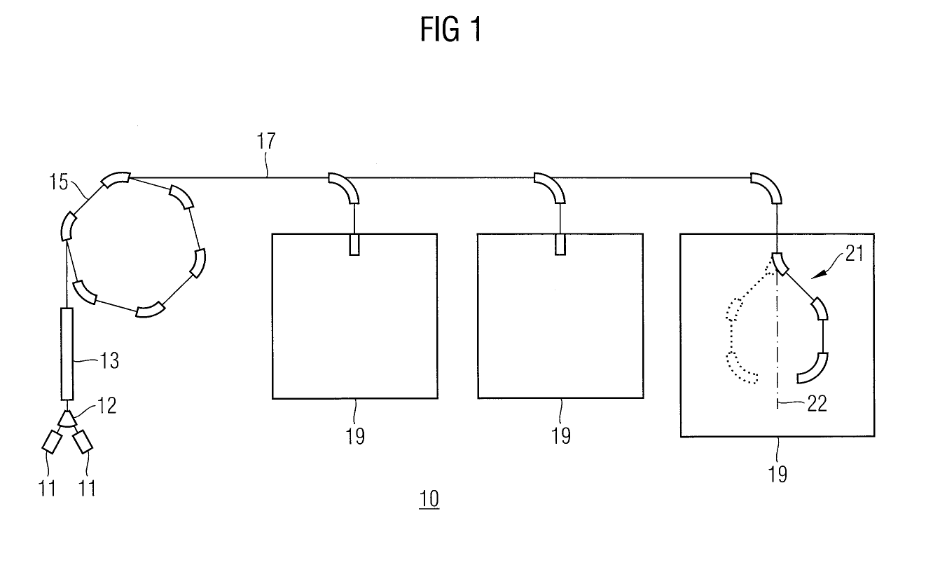

FIG. 1 shows a schematic overview of the design of a particle therapy system 10. In a particle therapy system 10, an irradiation of a body (e.g., a tumor-diseased tissue) takes place using a particle beam.

Ions such as, for example, protons, helium ions, carbon ions or other particle types such as pions may be used as particles. Particles of this type may be generated in a particle source 11. If, as shown in FIG. 1, two particle sources 11 that generate two different ion types exist, the two different ion types may be switched between within a short time interval. A switching magnet 12 is used, for example, to switch between the two different ion types. The switching magnet is arranged between the ion sources 11 and a preaccelerator 13. In one embodiment, the particle therapy system 10 may be operated with protons and carbon ions at the same time.

The ions generated by the ion source 11 or one of the ion sources 11 and selected with the switching magnet 12 are accelerated in the preaccelerator 13 to a first energy level. The preaccelerator 13 is a linear accelerator (LINAC), for example. The particles are fed into an accelerator 15 (e.g., a synchrotron or cyclotron). The particles are accelerated to high energies (e.g., as used for irradiation) in the accelerator 15. After the particles leave the accelerator 15, a high energy beam transportation system 17 guides the particle beam to one or several irradiation rooms 19. In an irradiation room 19, the accelerated particles are directed at a body to be irradiated. Depending on the embodiment, this takes place from a fixed direction (e.g., in “fixed beam” rooms) or from different directions by way of a moveable gantry, which may be rotated about an axis 22.

The design shown in FIG. 1 corresponds to a known particle therapy system 10. A particle therapy system 10 of this type may be developed such that the present embodiments may be used.

The present embodiments may however also be used in other particle therapy systems than those shown here. Radiation therapy systems operating with x-rays may also be used, provided the x-ray beams are created such that further photons develop in the target object as a result of interaction.

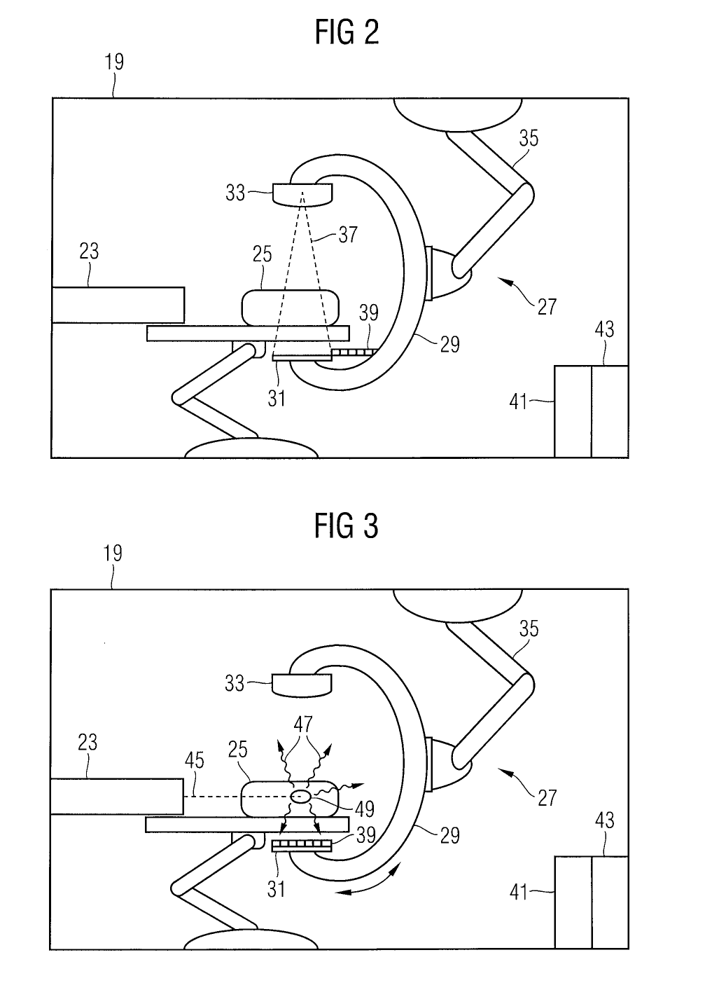

The present embodiments are described in more detail with the aid of the following FIGS. 2 to 4.

FIG. 2 shows a schematic representation of the design of an irradiation room 19, in which a particle beam may escape from a nozzle 23 and strike a target object 25 to be irradiated.

An imaging apparatus 27 is also arranged in the room. In the embodiment shown in FIG. 2, the imaging apparatus is a C-arm 29 with an x-ray detector 31 and an x-ray source 33. The C-arm 29 may be moved by way of a robot arm 25 suspended from the ceiling.

FIG. 2 shows the imaging apparatus 27 in the imaging mode. In the imaging mode, the x-ray source 33 is activated, and an image of the interior of the target object 25 to be irradiated may be recorded. This may take place, for example, prior to an irradiation session for the precise positioning of the target object 25 or also during the irradiation for recording and monitoring the current position of the target object 25. This is illustrated in FIG. 2 with diagnostic x-rays 37, which start from the x-ray source 33 and strike the x-ray detector 31. An adjustment apparatus 39, which may be moved in a controlled fashion upstream of the x-ray detector 31, is located in a position not in the radiation path of the diagnostic x-rays 37.

FIG. 2 illustrates a control apparatus 41 for controlling the imaging apparatus 27. The components of the imaging apparatus 27 may be positioned and activated with the control apparatus. FIG. 2 also illustrates an evaluation apparatus 43, with which the measurement data generated by the imaging apparatus 27 may be evaluated and further processed.

FIG. 3 shows the imaging apparatus 27 in a therapy monitoring mode. A particle beam 45 is activated, and radioactive isotopes are produced inside the target object 25 by interaction with the particle beam 45. The radioactive isotopes break up, and photons 47 are generated and emitted from the target object 25. The control apparatus 41 controls the imaging apparatus 27 such that the x-ray detector 31 is read out without activating the x-ray source 33. Information about the photons 47 produced in the target object 25 (e.g., spatial distribution and/or intensity of the photons 47) may be determined in this way. The evaluation apparatus 41 may at least partially reconstruct the deposited dose distribution 49 in the target object 25 from this determined information.

In the therapy mode, the adjustment apparatus 39 is arranged upstream of the x-ray detector 31. A scatter grid or an absorber plate may be used, for example, as an adjustment apparatus 39, so those photons that are not to be guided back for an interaction of the therapy beam 45 with the target beam 25 or strike the x-ray detector 31 from a “false” direction, may be filtered in an improved fashion. The therapy mode may be a single photon emission computed tomography (SPECT) recording mode.

By moving the imaging apparatus 27 during the irradiation and/or immediately after the irradiation, an improvement in the reconstruction of the recorded photons may be achieved, since photons 47 may be recorded from different directions. The movement is illustrated in FIG. 3 by the double arrow.

The recording of the photons 47 produced in the target object enables, for example, a subsequent evaluation of the deposited dose 49. The recording of the photons 47 may also be used online during an irradiation in order, for example, to halt an irradiation if a dose deposition is determined outside of a planned region.

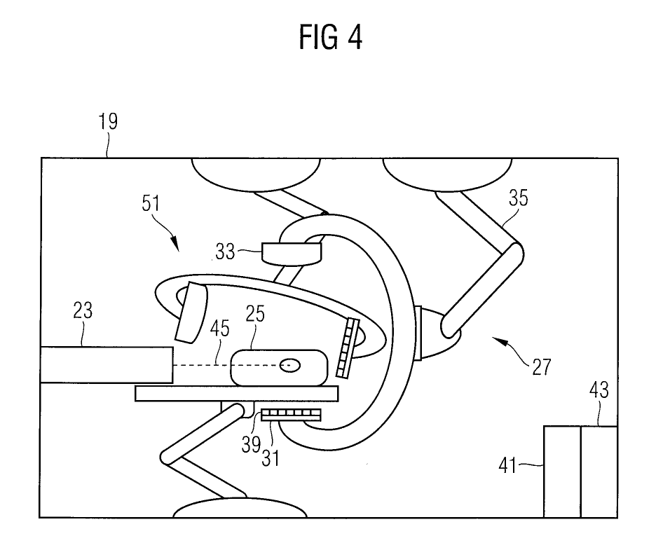

FIG. 4 shows one embodiment of a radiation therapy apparatus with a first imaging apparatus 27 and a second imaging apparatus 51 configured similarly. Both imaging apparatuses 27, 51 may be used and operated in the manner described above with the aid of FIG. 2 and FIG. 3.

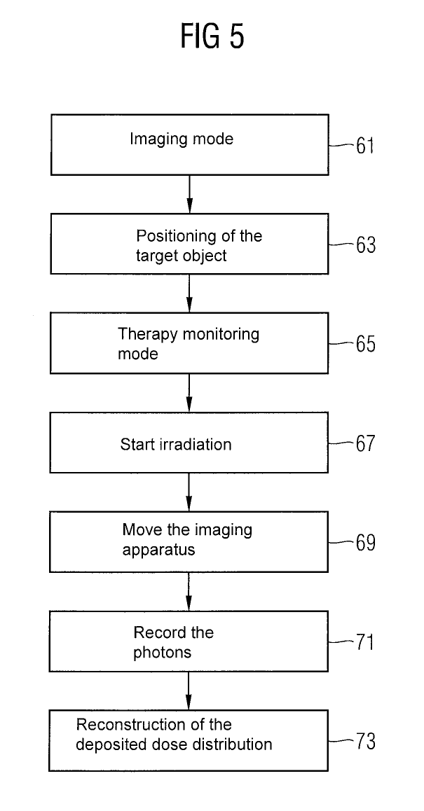

FIG. 5 illustrates a flow chart of monitoring an irradiation of a target object.

At act 61, a target object to be irradiated is positioned in an irradiation room for irradiation purposes, with an imaging mode of the imaging apparatus being activated. The imaging apparatus is used to produce an image of the inside of the target object, which is used when locating the correct position and during the positioning of the target object relative to the therapy beam (act 63).

The imaging apparatus is switched from the imaging mode into a therapy monitoring mode (act 65). The irradiation is started (act 67). During the irradiation, the imaging apparatus may be moved to different positions (act 69) in order to be able to record photons from more than one direction. Photons, which are produced in the target volume by interaction of the therapy beam with the target volume, are recorded with the x-ray detector (act 71) during the irradiation and immediately after the irradiation.

The recorded photons may be used to control the irradiation system. The recorded photons may however also be used to at least partially reconstruct the dose distribution deposited by the therapy beam (act 73).

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A radiation therapy apparatus comprising:
a source for providing a therapy beam that is directed from the source to a target object to be irradiated;
an imaging apparatus including an x-ray source and an x-ray detector, wherein the x-ray source and the x-ray detector are arranged such that diagnostic x-rays are directed through the target object to be irradiated onto the x-ray detector from the x-ray source; and
a controller for the imaging apparatus, the controller being configured to read out the x-ray detector during an irradiation of the target object with the therapy beam in order to record photons that develop within the target object as a result of the interaction of the therapy beam with the target object.

2. The radiation therapy apparatus as claimed in claim 1, further comprising:
an evaluation apparatus that is configured to reconstruct, at least partially, a dose distribution deposited in the target object by the therapy beam from the photons recorded with the x-ray detector.

3. The radiation therapy apparatus as claimed in claim 1, wherein the controller is configured to read out the x-ray detector without activating the x-ray source at the same time.

4. The radiation therapy apparatus as claimed in claim 1, wherein the imaging apparatus is positioned at different positions relative to the target object.

5. The radiation therapy apparatus as claimed in claim 4, further comprising a positioning apparatus to position the imaging apparatus.

6. The radiation therapy apparatus as claimed in claim 1, wherein the controller is configured to control the imaging apparatus such that the position of the radiation detector is changed during the recording of the photons during an irradiation of the target volume.

7. The radiation therapy apparatus as claimed in claim 1, further comprising another imaging apparatus including another x-ray source and another x-ray detector.

8. The radiation therapy apparatus as claimed in claim 1, wherein the x-ray detector comprises an adjustment apparatus for modifying the photons striking the x-ray detector, and
wherein the adjustment apparatus is arranged reversibly and is automatically controlled upstream of the x-ray detector.

9. A method for monitoring an irradiation of a target object, the method comprising:
irradiating the target object with a therapy beam and generating photons in the target object as a result of the interaction of the therapy beam with the target object;
recording the photons using an x-ray detector of an imaging apparatus, the imaging apparatus including an x-ray source and the x-ray detector for x-raying the target object with an x-ray beam; and
evaluating the recorded photons.

10. The method as claimed in claim 9, wherein recording the photons comprises recording the photons with the x-ray detector of the imaging apparatus without activating the x-ray source.

11. The method as claimed in claim 9, wherein evaluating the recorded photons comprises reconstructing, at least partially, a dose distribution deposited in the target object by the therapy beam from the recorded photons.

12. The method as claimed in claims 9, wherein the x-ray detector is moved to different positions during the recording of the photons.

13. The method as claimed in claims 9, further comprising modifying the photons striking the x-ray detector with an adjustment apparatus arranged upstream of the x-ray detector in order to record the photons,
wherein the adjustment apparatus is arranged reversibly in an automatically controlled manner.

14. The method as claimed in claim 13, wherein the x-ray detector is operable in an imaging mode, in which the x-ray detector interacts with the x-ray source in order to record image data of the target object to be irradiated, and a therapy monitoring mode, in which the x-ray detector records the photons produced in the target object by interaction with the therapy beam without interaction with the x-ray source.

15. The radiation therapy apparatus as claimed in claim 2, wherein the controller is configured to read out the x-ray detector without activating the x-ray source at the same time.

16. The radiation therapy apparatus as claimed in claim 2, wherein the controller is configured to control the imaging apparatus such that the position of the radiation detector is changed during the recording of the photons during an irradiation of the target volume.

17. The radiation therapy apparatus of claim 5, wherein the positioning apparatus is a robot arm.

18. The method as claimed in claim 10, wherein evaluating the recorded photons comprises reconstructing, at least partially, a dose distribution deposited in the target object by the therapy beam from the recorded photons.

19. The method as claimed in claims 11, further comprising modifying the photons striking the x-ray detector with an adjustment apparatus arranged upstream of the x-ray detector in order to record the photons,
wherein the adjustment apparatus is arranged reversibly in an automatically controlled manner.

20. The method as claimed in claims 12, further comprising modifying the photons striking the x-ray detector with an adjustment apparatus arranged upstream of the x-ray detector in order to record the photons,
wherein the adjustment apparatus is arranged reversibly in an automatically controlled manner.
